(19) United States
(12) Patent Application Publication
(10) Pub. No.: US 2024/0410864 A1
(43) Pub. Date: Dec. 12, 2024

(54) METHOD FOR DETECTING AND/OR QUANTIFYING MOOD DISORDER AND/OR IMPROVEMENTS OF THE MOOD DISORDER STATUS USING CONJUGATED BILE ACIDS AS BIOMARKER AND IMPROVED METHODS AND COMPOSITIONS THEREOF

(71) Applicant: SOCIETE DES PRODUITS NESTLE S.A., Vevey (CH)

(72) Inventors: FRANCOIS-PIERRE MARTIN, Vuisternens-devant-Romont (CH); GABRIELA BERGONZELLI DEGONDA, Bussigny (CH); ORNELLA COMINETTI ALLENDE, Denges (CH)

(21) Appl. No.: 18/694,904

(22) PCT Filed: Sep. 23, 2022
(86) PCT No.: PCT/EP2022/076515

§ 371 Date: Mar. 22, 2024

(30) Foreign Application Priority Data
Sep. 24, 2021 (EP) 21198767.2

(51) Int. Cl. G01N 30/88; A61K 31/198; A61K 35/00; A61K 35/741; G01N 30/02; G01N 30/72; G01N 33/53; H01J 49/00

(52) U.S. Cl. CPC G01N 30/88; A61K 31/198; A61K 35/741; G01N 30/7206; G01N 30/7233; G01N 33/5308; H01J 49/004; A61K 2035/115; G01N 2030/025; G01N 2030/027; G01N 2030/8813; G01N 2800/304

(57) ABSTRACT

The present invention relates to the use of glycine-conjugated bile acids as biomarker for detecting and/or quantifying improvements of the mood disorder status and/or excessive emotional reaction of a subject. It also relates to a method for detecting and/or quantifying mood disorder, improvements of the mood disorder status and/or excessive emotional reaction of a subject, in particular for monitoring the progress of an intervention to treat or ameliorate a mood disorder status and/or excessive emotional reaction in a subject, wherein the intervention comprises the administration of a probiotic. It also relates to an improved method to treat or ameliorate a mood disorder status and/or excessive emotional reaction in a subject in need, comprising administering to the subject, an effective amount of a composition combining a probiotic with glycine or derivative thereof.

The present invention relates to the use of glycine-conjugated bile acids as biomarker for detecting and/or quantifying improvements of the mood disorder status and/or excessive emotional reaction of a subject. It also relates to a method for detecting and/or quantifying mood disorder, improvements of the mood disorder status and/or excessive emotional reaction of a subject, in particular for monitoring the progress of an intervention to treat or ameliorate a mood disorder status and/or excessive emotional reaction in a subject, wherein the intervention comprises the administration of a probiotic. It also relates to an improved method to treat or ameliorate a mood disorder status and/or excessive emotional reaction in a subject in need, comprising administering to the subject, an effective amount of a composition combining a probiotic with glycine or derivative thereof.

According to a fact sheet published by the World Health Organization (WHO) in 2018, more than 300 million people worldwide suffer from depression (GBD 2017 Disease and Injury Incidence and Prevalence Collaborators. (2018). Global, regional, and national incidence, prevalence, and years lived with disability for 354 diseases and injuries for 195 countries and territories, 1990-2017: a systematic analysis for the Global Burden of Disease Study 2017. The Lancet). Depression, one mood disorder, is a common illness that is different from normal changes in mood and short-lived emotional responses to challenges in everyday life. However, subclinical depression, a milder mood disorder may also affect quality of life.

Mood disorders can have severe effects for the concerned individual and for the persons the affected individual is interacting with on a regular basis. Typical consequences are poor performance at work or in school, decreased social interaction, personal suffering and a negative influence on relationships with friends or family.

In the worst case, mood disorder can lead to suicide. Close to 800 000 people die due to suicide every year with suicide being the second leading cause of death in 15-29-year-olds (Suicide worldwide in 2019: global health estimates. Geneva: World Health Organization; 2021. Licence: CC BY-NC-SA 3.0 IGO).

Mood disorders appear to be more prevalent in women than in men (Journal of the American Medical Association, 2003; Jun. 18; 289 (23): 3095-105) perinatal period, and post-menopause being particular susceptible moments. Also 1.9 million children are diagnosed with depression. Notably, mood disorders may also lead to other diseases later on. It is known, for example, that mood disorders result in a greater risk to develop coronary artery disease.

Mood disorders can usually be treated successfully today. For example, mood disorders can be treated by exercise or talking therapy, ideally guided by a psychologist. Psychotherapy, for example a cognitive behavioral therapy, is an option. As medicaments antidepressants are used successfully today. Often combinations of the above referenced approaches are used in the framework of a combination therapy. Recent scientific work has revealed that the probiotic Bifidobacterium longum (BL) NCC3001 reduces depression scores (Gastroenterology 2017; 153:448-459) in patients with irritable bowel syndrome.

Doctors diagnose mood disorders today by talking with the patient and by screening for typical symptoms. Treatment responses should be measured today by systematically monitoring patients' responses using validated self-rated scales (J Clin Psychiatry. 2013 July; 74 (7)).

On one side, it would be desirable to have available a biochemical tool that allows it to detect mood disorders and/or to assess the success of a therapy to treat or ameliorate mood disorders, and also to improve such therapy.

The present inventors have addressed these needs.

Any reference to prior art documents in this specification is not to be considered an admission that such prior art is widely known or forms part of the common general knowledge in the field.

The objective of the present invention was, hence, to improve the state of the art and in particular to provide a biochemical tool that allows it to diagnose mood disorders or improvements of the mood disorder status and/or excessive emotional reaction of a subject, or to at least provide a useful alternative. It also aimed to an improve method to treat or ameliorate a mood disorder status and/or the excessive emotional reaction in a subject.

The inventors were surprised to see that the objective of the present invention could be achieved by the subject matter of the independent claims. The dependent claims further develop the idea of the present invention.

Accordingly, in one aspect, the present invention provides a biomarker that can be measured in blood, wherein the biomarker is a conjugated bile acid, in particular a glycine-conjugated bile acid.

In another aspect, the present invention provides further the use of conjugated bile acids as biomarker for detecting and/or quantifying improvements of the mood disorder status and/or excessive emotional reaction of a subject.

In a further aspect, the present invention provides a method for detecting and/or quantifying mood disorders, improvements of the mood disorder status and/or excessive emotional reaction of a subject, comprising the assessment of the level of at least one conjugated bile acid in a body sample obtained from a subject to be tested, and comparing the subject's conjugated bile acid level to a predetermined reference value, wherein
i) an increased level of conjugated bile acid cholic acid (CA) and/or chenodeoxycholic acid (CDCA), in the sample compared to the predetermined reference value and/or
ii) a decreased level of conjugated bile acid ursodeoxycholic acid (UDCA), in the sample compared to the predetermined reference value,
indicates an improvement of the mood disorder status and/or excessive emotional reaction of the subject.

In a last aspect, the present invention provides an improved method to treat or ameliorate a mood disorder status and/or excessive emotional reaction in a subject comprising administration to the subject in need, of an effective amount of a composition combining a probiotic with glycine or a derivative thereof.

As used in this specification, the words “comprises”, “comprising”, and similar words, are not to be interpreted in an exclusive or exhaustive sense. In other words, they are intended to mean “including, but not limited to”.

As used herein, “treat”, “treating” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s). As used herein, “prevent”, “preventing”, “prevention”, or “prophylaxis” of a disease or disorder means preventing that a disease or disorder occurs in subject.

The terms “effective amount” or “therapeutic amount” are intended to mean that amount of a substance that will elicit the physiological response of a tissue, a system, animal or human that is being sought by a researcher, veterinarian, medical doctor or other clinician. The term “prophylactically effective amount” is intended to mean that amount of a substance that will prevent or reduce the risk of occurrence of the biological or medical event that is sought to be prevented in a tissue, a system, animal or human by a researcher, veterinarian, medical doctor or other clinician.

For the purpose of the present invention the term “mood disorder” shall be understood to include mental health problem that primarily affects a person's emotional state. It includes affective disorders/disturbances such as manic (elevated, expansive, or irritable mood with hyperactivity, pressured speech, and inflated self-esteem) or depressive (dejected mood with disinterest in life, “empty” feelings, loss of interest or pleasure, sadness, changes in appetite or weight, lack of or decreased energy, sleep disturbance, agitation, and feelings of worthlessness or guilt, helplessness, difficulty in thinking, concentrating, or making decision, hopelessness, tiredness, fatigue, memory difficulties, tearfulness) episodes, and often combinations of the two.

The term “mood” refers to a state or quality of feeling (an emotional state) at a particular time. Moods differ from simple emotions in that they are less specific, less intense, and less likely to be triggered by a particular stimulus or event. Clinical depression and bipolar disorder are examples of mood disorders (i.e., long-term disturbances of mood). Mood disorders are a group of diagnoses in the classification system of the Diagnostic and Statistical Manual of Mental Disorders (DSM) where disturbances in mood are the main underlying feature. Non-limiting examples of depressive disorders include severe depression like major depression disorders and subclinical depression which is a mild to moderate mood disorder, disruptive mood dysregulation disorder, major depressive disorder, single and recurrent episodes, persistent depressive disorder (Dysthymia), Seasonal affective disorder (SAD), premenstrual dysphoric disorder, substance/medication-induced depressive disorder, depressive disorder due to another medical condition, other specified depressive disorder or unspecified depressive disorder.

For the purpose of the present invention the term “excessive emotional reaction” includes emotional dysregulation characterized by excessive fear, anxiety, anger, or sadness. Non-limiting examples of anxiety disorders includes separation anxiety disorder, selective mutism, specific phobia, social anxiety disorder (social phobia), panic disorder, panic attack, agoraphobia, generalized anxiety disorder, substance/medication-induced anxiety disorder, anxiety disorder due to another medical condition, other specified anxiety disorder or unspecified anxiety disorder. It can also refer to stress, feeling of excessive stress, irritability, restlessness or excessive worry over physical health.

A mood disorder can alternatively be a secondary condition caused by an underlying medical condition selected from the group consisting of a neurological disorder, a metabolic disorder, a function gastrointestinal disorder, an endocrine disease, a cardiovascular disease, a pulmonary disease, a cancer, an autoimmune disease, and combinations thereof. For example, the mood disorder can be one or more depressive symptoms arising from the underlying medical condition.

The present inventors have shown that conjugated bile acids can be used as a biomarker for detecting and/or quantifying improvements of the mood disorder status and/or excessive emotional reaction of a subject.

Without wishing to be bound by theory, the inventors presently believe that circulating conjugated bile acids, in particular glycine-conjugated bile acids might be a readout indicative of a shift in protein and aromatic amino acid metabolism by the gut microbiota, and therefore might directly or indirectly describe probiotic-induced gut-brain metabolic interactions associated with the improvement of the mood disorder status. Also, glycine-conjugated bile acids, cholic acid, chenodeoxycholic acid are amongst the most abundant species involved in emulsification of dietary fats, and are increased with probiotics. Several mode of actions could be mediated by the modulation of the bile acids composition exerted by probiotics such as BL NC3001, such as (i) influencing the bioavailability of glycine for normal neurotransmitter metabolism, and for which supplementation is associated with improved mood and cognition, and sleep (Bannai et al. Front Neurol. 2012; 3:61), (ii) modulation of emulsification, absorption and bioavailability of dietary fats, including PUFAs, to cross blood brain barrier and contribute to brain function involved in mood, depression and anxiety (Kuan-Pin Su et al. Clin Psychopharmacol Neurosci. 2015 August; 13 (2): 129-137), (iii) Modulation of the synthesis of Oleyl and palmitoyl ethanolamines lipids in the enterocytes, know to modulate Anxiety- and depressive-like behaviour as well as pain perception and sensory transduction (Hill M N et al., Psychoneuroendocrinology. 2009 September; 34 (8): 1257-62. And Coppola M, et al. Med Hypotheses. 2014 May; 82 (5): 507-11)., (iv) and by direct interaction with specific receptor such as FXR or, TGR5 receptors (Kiriyama Y, et al. Biomolecules. 2019).

The present inventors have carried out the studies presented herein using an intervention with the probiotic BL NCC3001 as an example. Consequently, for the purpose of the present invention the probiotic may be Bifidobacterium longum, for example BL NCC3001.

Also companion animals can suffer from mood disorders. For the purpose of the present invention, a companion animal is an animal kept primarily for a person's company, entertainment or as an act of compassion. Typical examples for companion animals are cats or dogs; but also rabbits; ferrets; pigs; rodents, such as gerbils, hamsters, chinchillas, rats, mouse and guinea pigs; or birds. When, for example, dogs are depressed, they often appear withdrawn, lose interest to play, and/or appear lethargic or sad. Sometimes, they will eat and/or drink less than usual which might result in a variety of physical illnesses. As a result, today also companion animals are treated for mood disorders. Hence, in one embodiment of the present invention, the subject may be a human or a companion animal such as a cat or a dog.

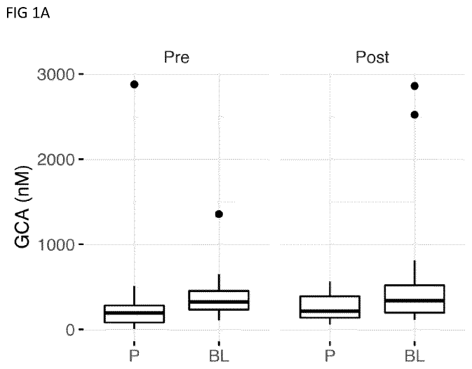

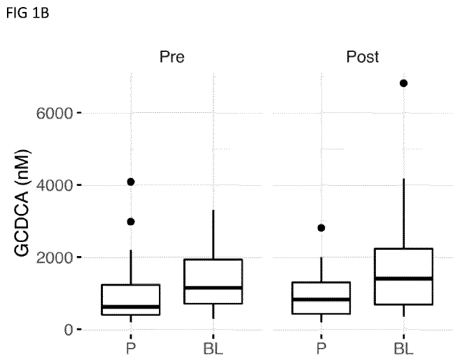

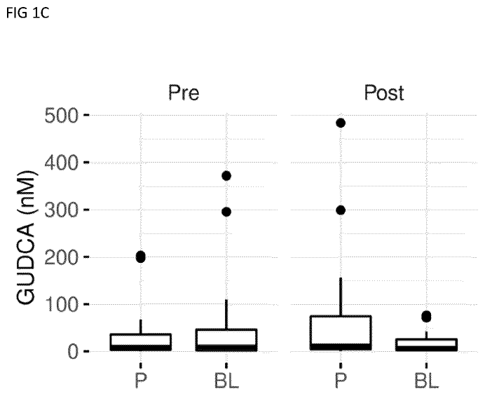

FIG. 1A to C show blood concentrations in the markers (A) GCA, (B) GCDCA, (C) GUDCA, reported as a boxplot depicting groups of concentration data through their quartiles.

Consequently, the present invention relates in part to a biomarker, wherein the biomarker is a conjugated bile acid. The conjugated bile acid is a taurine or glycine conjugate of bile acids, preferably a glycine-conjugated bile acid.

In an embodiment the bile acid is selected from cholic acid (CA), chenodeoxycholic acid (CDCA), Ursodeoxycholic acid (UDCA), and their glycine-conjugated form.

Biomarkers are well known to people skilled in the art. They are usually understood as a characteristic that is objectively measured and evaluated as an indicator of normal biological processes, pathogenic processes, or responses to an intervention. Further guidance can be obtained from Curr Opin HIV AIDS. 2010 November; 5 (6): 463-466.

The present invention also relates to the use of at least one conjugated bile acid as a biomarker for detecting and/or quantifying improvements of the mood disorder status and/or excessive emotional reaction of a subject. Accordingly, conjugated bile acids may be used as a biomarker for detecting mood disorders.

In a preferred embodiment, the mood disorder is mild to severe. The Hospital Anxiety and Depression Scale (HADS) can be used to measure the level of mood disorder. The HADS is a 14-item self-report measure, with seven items forming a depression subscale and another seven items measuring anxiety (Zigmond & Snaith, 1983). Each item is rated on a four-point scale, ranging from 0 to 3, with 3 indicating higher symptom frequency. Total scores for each subscale range from 0 to 21, categorized as: normal (0-7), mild (8-10), moderate (11-14) or severe (15-21) (The Hospital Anxiety and Depression Scale, Occupational Medecine 2014, 64:393-394).

Conjugated bile acids may further be used for detecting and/or quantifying improvements of the mood disorder status.

Conjugated bile acids may further be used for detecting and/or quantifying improvements of the emotional reaction of a subject resulting from its mood disorder status. For example, the authors of Gastroenterology 2017; 153:448-459 describe that a change in engagement of the amygdala correlated with a change in mood disorder scores. The amygdala plays a primary role in emotional responses, so that it can be concluded that an improvement of the mood disorder status will correspond to an improvement of the emotional reaction of a subject resulting from its mood disorder status.

The subject matter of the present invention further relates to a method for detecting and/or quantifying improvements of the mood disorder status and/or excessive emotional reaction of a subject, comprising
assessing the level of at least one conjugated bile acid in a body sample obtained from a subject to be tested, and
comparing the subject's conjugated bile acid level to a predetermined reference value,
wherein:
i) an increased level of conjugated bile acid CA and/or CDCA, in the sample compared to the predetermined reference value and/or
ii) a decreased level of conjugated bile acid UDCA, in the sample compared to the predetermined reference value,
indicates an improvement of the mood disorder status and/or excessive emotional reaction of the subject.

The subject matter of the present invention further relates to a method for detecting mood disorders in a subject, comprising assessing the level of at least one conjugated bile acid in a body sample obtained from a subject to be tested, and
comparing the subject's conjugated bile acid level to a predetermined reference value,
wherein:
i) an increased level of conjugated bile acid CA and/or CDCA, in the sample compared to the predetermined reference value and/or
ii) a decreased level of conjugated bile acid UDCA, in the sample compared to the predetermined reference value,
indicates an improvement of the mood disorder status and/or excessive emotional reaction of the subject.

The method of the present invention has the advantage that it allows to diagnose mood disorders based on the concentration of a biomarker or the change of the concentration of a biomarker in a body sample. It also allows to control the success of a treatment of mood disorders in a subject. Such a biochemical method can, hence, be a valuable tool to assist doctors to accurately diagnose mood disorders and/or to follow the success of the treatment they prescribe, while they would otherwise largely have to only rely on subjective questionnaires and patients' description of their symptoms. Also, the method of the present invention will be very valuable to help subjects that are unable to communicate clearly and suffer from mood disorders, for example companion animals.

The method of the present invention compares a level of at least one conjugated bile acid in a body sample obtained from a subject to be tested with a reference value.

For example, when aiming to detecting and/or quantifying improvements of the mood disorder status and/or excessive emotional reaction of a subject, it may be preferred if the reference value was also obtained from the subject to be treated.

Hence, for the method of the present invention, the predetermined reference value may have been obtained previously from the same subject. This has the advantage that an increased level of at least one conjugated bile acid selected from the group consisting of GCA and GCDCA, and/or a decreased level of at least one conjugated bile acid such as GUDCA can be reliably measured for an individual by comparing the current conjugated bile acid level to a previous conjugated bile acid level.

Alternatively, the predetermined reference value may be based on an average conjugated bile acid level in the same body sample in a control population. This has the advantage that the measured conjugated bile acid level of an individual can be compared to a standard that is generally applicable, so that the conjugated bile acid level of an individual can be compared to a general average. This allows for an easy comparison of many measurements in many individual patients. It also allows for a quick assessment by making one test only, as there is no need for a previous test to obtain an individual reference value.

The analysis of the at least one conjugated bile acid level in the body sample can be carried out by any suitable method known to the person skilled in the art. The present inventors have used mass spectrometry. Hence, in one embodiment of the present invention, the level of the biomarker in the sample and in the reference may be determined by mass spectrometry. For an increased speed, accuracy and reduction of noise, mass spectrometry may be coupled with a chromatographic step preceding the mass spectrometry. For example, the level of the biomarker in the sample and in the reference may be determined by ultra-performance liquid chromatography coupled to tandem mass spectrometry. Further, for example the level of the biomarker in the sample and in the reference may be determined by gas chromatography coupled to tandem mass spectrometry. For example, the quantitative measurement of the conjugated bile acid level in samples may be carried out using both ultra-performance liquid chromatography coupled to tandem mass spectrometry (UPLC-MS/MS) and/or gas chromatography time-of-flight mass spectrometry (GC-TOFMS).

Advantageously, in order to ensure optimal comparability of reference value and the conjugated bile acid level obtained from the body sample, both, the reference value and the present conjugated bile acid level may be obtained from the same body sample. Hence, the predetermined reference value may be based on a conjugated bile acid level obtained from the same body sample as the level of conjugated bile acid in a body sample obtained from a subject to be tested.

The method of the present invention may be used to monitor the success of a mood disorder treatment. In order to do this, it may be preferred to be able to compare current conjugated bile acid levels to a conjugated bile acid level obtained from the subject who is being treated before the treatment was started. Hence, for example, the subject's predetermined reference value may be obtained from a body sample that was collected from the subject before an intervention to treat or ameliorate a mood disorder status and/or excessive emotional reaction started.

In order to be able to assess further improvements in mood disorder status after an invention has already started, it may still be preferred to have available a reference obtained from the subject that is being treated. Hence, for example, the subject's predetermined reference value may be obtained from a body sample that was collected from the subject during an intervention to treat or ameliorate a mood disorder status and/or excessive emotional reaction, but at least one week, for example at least two weeks, at least four weeks, or at least six weeks, before the body sample is obtained from the subject. This has the advantage that a continuous progress in the treatment of the mood disorder can continuously be monitored.

Generally, an increase in the detected level of at least one conjugated bile acid selected from the group consisting of GCA and GCDCA and/or a decrease in the detected level of one conjugated bile acid GUDCA indicates an improvement of the mood disorder status and/or excessive emotional reaction of the subject. However, one advantage of the biomarker of the present invention is that the differences in biomarker concentration in the body sample that can be measured in a successful treatment are rather pronounced. Hence, for example, in the method of the present invention an increase and/or decrease in the conjugated bile acid level in the sample compared to the predetermined reference value of at least 10%, at least 20%, or at least 30% indicates an improvement of the mood disorder status and/or excessive emotional reaction of the subject.

The present inventors have found that typical body samples that may be used for the purpose of the present invention may be selected from the group consisting of whole blood, blood serum, and blood plasma.

Advantageously, both reference and the current conjugated bile acid level are both obtained from the same body sample, for example, both reference and the current conjugated bile acid level are both obtained from whole blood, both reference and the current conjugated bile acid level are both obtained from blood serum, or both reference and the current conjugated bile acid level are both obtained from blood plasma.

For example, from, blood, blood serum, or blood plasma about 5-10 ml may be collected. A large enough sample size avoids that artifacts are generated. From these samples, about 20-100 μl may be used for further analysis.

Whole blood, blood serum and/or blood plasma have the advantage that the signal to noise ratio for the biomarker to be tested is particularly high. Irrespective of the chosen body sample, the method of the present invention has the advantage that obtaining such body fluids from a subject is a well-established procedure. The actual diagnosis method is then carried out in a body sample outside the body.

The method of the present invention is suitable to monitor the progress of any treatment of mood disorders. For example, the mood disorder treatment may be selected from the group consisting of exercise, talking therapy, psychotherapy, cognitive behavioral therapy, antidepressant administration, nutritional intervention for example with probiotics, and combinations thereof.

Probiotics have been found to have an effect on the symptoms of mood disorder (Neuropsychobiology. 2019 Feb. 13:1-9) (Gastroenterology 2017; 153:448-459). These probiotics can help to treat a range of mental health conditions, including mood disorders. Without wishing to be bound by theory, the present inventors currently believe that this effect is seen, because of the gut-brain axis, a strong connection between gastrointestinal tract and brain. Hence, in one embodiment of the present invention, the method is for monitoring the progress of an intervention to treat or ameliorate a mood disorder status and/or excessive emotional reaction in a subject, wherein the intervention comprises the administration of a probiotic.

In a last aspect, the present invention provides an improved method to treat or ameliorate a mood disorder status and/or excessive emotional reaction in a subject comprising administering to the subject in need, an effective amount of a composition combining a probiotic with glycine or a derivative thereof.

In a preferred embodiment, the mood disorder is mild to severe.

The composition can be administered to improve mood disorder status and/or excessive emotional reaction of a subject. Accordingly, some embodiments of the methods comprise diagnosing the subject, before initiating administration of the composition.

In an embodiment, an improved mood may comprise one or more of a decreased depressive level, a decreased anxiety level, a decreased stress level, an increased perceived energy level (“vitality”), a more positive emotional state, an increased self-esteem, a reduced amount and/or a reduced intensity of negative thoughts and/or negative tensions, a reduced risk of mood swings, or retention of a positive mood. Further in this regard, the composition can be administered to reduce anxiety and/or to reduce stress in an individual in need thereof. The method can comprise identifying the individual as being in need of reduced anxiety and/or reduced stress.

As noted above, the composition can be administered to modulate excessive emotional distress (e.g. prevent or treat a phobia). Accordingly, some embodiments of the methods of modulating excessive emotional distress disclosed herein comprise diagnosing the individual having excessive emotional distress, e.g., before initiating administration of the composition.

In an embodiment, glycine is provided in the form of L-glycine or in the form of sources rich in glycine, such as protein, peptide, or glycine derivates.

Non-limiting examples of suitable glycine functional derivatives include D-Allylglycine; N-[Bis(methylthio)methylene] glycine methyl ester; Boc-allyl-Gly-OH (dicyclohexylammonium) salt; Boc-D-Chg-OH; Boc-Chg-OH; (R)-N-Boc-(2′-chlorophenyl)glycine; Boc-L-cyclopropylglycine; Boc-L-cyclopropylglycine; (R)-N-Boc-4-fluorophenylglycine; Boc-D-propargylglycine; Boc-(S)-3-thienylglycine; Boc-(R)-3-thienylglycine; D-a-Cyclohexylglycine; L-a-Cyclopropylglycine; N-(2-fluorophenyl)-N-(methylsulfonyl)glycine; N-(4-fluorophenyl)-N-(methylsulfonyl)glycine; Fmoc-N-(2,4-dimethoxybenzyl)-Gly-OH; N-(2-Furoyl)glycine; L-a-Neopentylglycine; D-Propargylglycine; sarcosine; Z-a-Phosphonoglycine trimethyl ester, and a mixture thereof. In an embodiment, the glycine may be provided in a dipeptide, such as N-acetylcysteinylglycine or cysteinylglycine.

The glycine (GLY) or functional derivative thereof can be administered in an amount of about 0.1-100 milligram (mg) of glycine or functional derivative thereof per kilogram (kg) of body weight of the subject. In embodiments, glycine, or analogue thereof, may also be formulated for administration in a dose of between about 10 μmol/kg body weight to about 500 mmol/kg body weight, preferably 10 μmol/kg body weight to 100 mmol/kg body weight, more preferably 10 μmol/kg body weight to 10 mmol/kg body weight, more preferably 50 μmol/kg body weight to 2 mmol/kg body weight.

In an embodiment, the probiotic of the invention may be Bifidobacterium longum, B. infantis, B. animalis ssp. lactis, or B. breve. Most preferably, it is B. longum, for example B. longum subsp. longum, B. longum subsp. infantis, or B. longum subsp. suis, preferably B. longum subsp. longum. The B. longum subsp. longum can be selected from B. longum ATCC BAA-999, B. longum ATCC 15707, and B. longum CNCM I-2618. Most preferably, it is B. longum ATCC BAA-999 (NCC3001).

B. longum ATCC BAA-999 was deposited by the Assignee of the present application as NCC 3001 on Jan. 29, 2001 at the Institut Pasteur, 28 rue du Docteur Roux, F-75024 Paris Cedex 15, France. All restrictions upon public access to the deposits will be irrevocably removed upon grant of a patent on this application, and the deposits will be replaced if viable samples cannot be dispensed by the depository.

The B. longum ATCC BAA-999 may be cultured according to any suitable method. B. longum ATCC BAA-999 may be added to a food product in a freeze-dried or spray-dried form, for example, to form the composition.

It is clear to those skilled in the art that an ideal dose will depend on the subject to be treated, its health condition, sex, age, or weight, for example, and the route of administration. The dose to be ideally used will consequently vary but can be determined easily by those of skill in the art.

However, generally, it is preferred if the composition of the present invention comprises between 10⁶ and 10¹⁰ cfu and/or between 10⁶ and 10¹⁰ cells of B. longum subsp longum per daily dose. It may also comprise between 10⁶ and 10¹¹ cfu and/or between 10⁶ and 10¹¹ cells of B. longum subsp longum per g of the dry weight of the composition. Alternatively, a daily dose of the composition preferably provides between 10⁴ and 10¹² cfu (colony forming units) of the B. longum e.g. ATCC BAA-999, more preferably from 10⁴ to 10¹¹ cfu, most preferably from 10⁴ to 10¹⁰ cfu. The composition may comprise between 10² and 10¹⁰ cfu, preferably 10² to 109 cfu, more preferably 10² to 10⁸ cfu of the B. longum, e.g ATCC BAA-999 per gram dry weight of the composition.

In the case of inactivated and/or non-replicating B. longum, e.g ATCC BAA-999, the composition can comprise between 10² and 10¹⁰ non-replicating cells of the B. longum per gram of dry weight of the composition, preferably 10³ to 10⁸ non-replicating cells per gram of dry weight of the composition, more preferably 10⁵ to 10⁸ non-replicating cells per gram of dry weight of the composition.

The composition can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

In each of the compositions and methods disclosed herein, the composition is preferably a food product or beverage product, including food additives, food ingredients, functional foods, dietary supplements, medical foods, nutraceuticals, oral nutritional supplements (ONS) or food supplements, or infant formula.

The compositions disclosed herein may be administered to the subject orally, enterally, or intraocularly, topically or inhalation. As such, non-limiting examples of the form of the composition include natural foods, processed foods, natural juices, concentrates and extracts, microcapsules, nano-capsules, liposomes, plasters, inhalation forms, nose sprays, nosedrops, eyedrops, sublingual tablets, and sustained-release preparations.

The compositions disclosed herein can use any of a variety of formulations for therapeutic administration. More particularly, pharmaceutical compositions can comprise appropriate pharmaceutically acceptable carriers or diluents and may be formulated into preparations in solid, semi-solid, liquid or gaseous forms, such as tablets, capsules, powders, granules, ointments, solutions, suppositories, injections, inhalants, gels, microspheres, and aerosols. As such, administration of the composition can be achieved in various ways, including oral, buccal, rectal, enteral and intratracheal administration. The active agent may be systemic after administration or may be localized by the use of regional administration, intramural administration, or use of an implant that acts to retain the active dose at the site of implantation.

In pharmaceutical dosage forms, the compounds may be administered as their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch;

with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Compositions intended for a non-human animal include food compositions to supply the necessary dietary requirements for an animal, animal treats (e.g., biscuits), and/or dietary supplements. The compositions may be a dry composition (e.g., kibble), semi-moist composition, wet composition, or any mixture thereof. In one embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement may require admixing or can be admixed with water or other diluent prior to administration to the animal.

Those skilled in the art will understand that they can freely combine all features of the present invention disclosed herein. In particular, features described for the biomarker of the present invention may be combined with the use of the present invention and the method of the present invention and vice versa. Further, features described for different embodiments of the present invention may be combined.

Although the invention has been described by way of example, it should be appreciated that variations and modifications may be made without departing from the scope of the invention as defined in the claims.

Furthermore, where known equivalents exist to specific features, such equivalents are incorporated as if specifically referred in this specification. Further advantages and features of the present invention are apparent from the figures and non-limiting examples.

### Example

### Methods

### Study Oversight

We conducted a randomized, double-blind, placebo-controlled, single center pilot study in patients with non-constipated irritable bowel syndrome (IBS) (Pinto-Sanchez et al., Gastroenterology 2017).

### Participants

We recruited adult patients with a diagnosis of non-constipated IBS (Rome III criteria (Longstreth G F, Thompson W G, Chey W D, et al. Functional bowel disorders. Gastroenterology 2006; 130 (5): 1480-91), and mild to moderate anxiety and/or depression scores based on the Hospital Anxiety and Depression (HAD) scale (Snaith R P, Zigmond A S. The HAD scale with the Irritability depression-anxiety scale and the Leeds situational anxiety scale manual. Published by GL assessment Ltd. 1994) (HAD-A or HAD-D score 8-14). Patients with a history of organic diseases, immune deficiency, major abdominal surgery, a psychiatric condition other than anxiety or depression, use of immunosuppressants, glucocorticosteroids, opioids, antidepressants or anxiolytics in regular doses, alcohol or illicit drug consumption, were excluded. Loperamide and laxatives were allowed as rescue medications. Other probiotics in any form were forbidden during the 1-month run-in period and the trial. Antibiotics were forbidden during the 3 months prior to the run-in period and the trial.

### Design of the Study

The study involved four hospital visits. At the screening visit, clinical history and symptoms were assessed and physical exam and complete bloodwork performed. At the second visit (week 0), the inclusion and exclusion criteria and symptoms were re-assessed, stool, urine and blood samples were collected, and an fMRI study performed.

The patients were then randomised to receive 42 sachets of either spray dried B. longum (1.0E+10 CFU/1 gram of maltodextrin powder) or placebo containing 1 gram of maltodextrin. Treatment products were indistinguishable in terms of package, color, taste and consistency. Patients were instructed to dissolve the content of the sachet in 100-200 ml of lactose-free milk, soy milk or rice milk, preheated to 20° Celsius. Patients were asked not to change their eating habits or fibre intake. Participants recorded the treatment intake, the empty sachets were used to assess the compliance at the third visit (week 6), where their symptoms were assessed, blood, urine and stool samples collected and fMRI test performed. Finally, patients' symptoms were re-assessed at a follow-up visit (week 10).

In addition to the regular hospital visits, Hospital anxiety and depression (HAD) scores were also assessed at 3 weeks of treatment following request of Health Canada. HAD questionnaires were provided to patients at Visit 1 and then mailed or e-mailed to the investigators.

### Study Endpoints

The primary endpoint was a reduction in anxiety and/or depression scores of ≥2 points on HAD scales (Longstreth G F, Thompson W G, Chey W D, et al. Functional bowel disorders. Gastroenterology 2006; 130 (5): 1480-91) at 6 weeks. This was based on the previously established mean clinically important difference for the anxiety and depression scores on the HAD scale of 1.3 and 1.4, respectively (Puhan M, Frey M, Buchi S, et al. The minimal important difference of the hospital anxiety and depression scale in patients with chronic obstructive pulmonary disease. Health Qual Life Outcomes. 2008; 6:46.). Secondary endpoints included improvement in anxiety and depression scores (HAD, continuous data), anxiety (State-Trait Anxiety Inventory, STAI), IBS global adequate relief, IBS symptoms, somatization, quality of life, changes in brain activation patterns (functional Magnetic Resonance Imaging, fMRI), serum inflammatory markers, neurotransmitters and BDNF, and plasma metabonomic and stool microbiota profiles.

### Randomization

The randomization sequence was performed using a computer program (Proc Plan, SAS, V. 9.1). A block randomization was stratified by gender and IBS status (diarrhea or mixed stool pattern). The codes were kept in sealed opaque envelopes allocated to patients according to strata. Each pack was assigned a number according to the randomization sequence. On recruitment, the patients were assigned into one of four strata and given the next consecutive randomization number available for that stratum. Treatment allocation was concealed from participants and study staff. Treatment products indistinguishable in terms of package, color, taste and consistency, were identified with two non-speaking codes per arm. Their identity was blind to subjects, investigators and support staff.

### Study Measurements

Anxiety and depression were assessed by the HAD-A and HAD-D sub-scores respectively. As an additional measure of anxiety we used the STAI (Gaudry E, Spielberger C D, Vagg P. Validation of state-trait distinction in anxiety distinction. Multivariate Behav Res 1975; 10:331-41) which assesses both stait and trait anxiety.

Brain activity was assessed by functional magnetic resonance imaging (fMRI) using General Electric 3-Tesla Discovery M R 750, whole body short bore scanner with 32 parallel receiver channels (General Electric, Milwaukee, WI). The 1-hour protocol included a seven minute T1 weighted structural scan, followed by four repetitions of a fearful face backward masking paradigm (Hall G B, Doyle K A, Goldberg J, et al. Amygdala engagement in response to subthreshold presentations of anxious face stimuli in adults with Autism Spectrum Disorders: preliminary insights. PloS One 2010; 5 (5): e10804) during four fMRI Blood Oxygen Level Dependent scans (He X, Yablonskiy D A.

Quantitative BOLD: mapping of human cerebral deoxygenated blood volume and oxygen extraction fraction: default state. Magn Reson Med 2007; 57:115-26) (BOLD EPI; TR/TE=2800/35 ms, flip angle=90°, 3 mm thick slices, no gap, field of view=24 cm, matrix=64×64). Pre-processing of MRI data was completed using Brain Voyager Q X Version 2.8.2, 32-bit (Brain Innovation, Maastricht, Netherlands). Anatomic and functional data were inspected and scans with artefacts or fMRI scans with movement greater than 5 mm in any of 6 planes were excluded from analysis. Anatomical scans were transformed into standard sagittal orientation, and underwent spatial normalization into standard Talaraich space. Slice scan time correction and 3D motion correction were carried out on the fMRI data and spatial smoothing applied using a Gaussian filter (FWHM=6 mm). Amygdala was selected as region of interest (ROI), initially derived from the WFUPick Atlas and refined according to anatomic landmarks on the full group average transformed T1 image.

Blood samples were collected after an overnight fast. After processing, the samples were stored at −80 C until assessment.

Metabonomic analysis was conducted in blood to measure specific panels of bile acids. The samples were extracted and prepared according to previously published methods (Xie, Zhong et al. 2013, Zhao, Ni et al. 2017). For bile acid analysis, all standards were obtained from Steraloids Inc. (Newport, RI) and TRC Chemicals (Toronto, ON, Canada), and 9 stable isotope-labeled bile acid standards were obtained. The calibration curve samples were prepared in the blank matrix and processed in the same way as real biological samples. An ultra-performance liquid chromatography coupled to tandem mass spectrometry (UPLC-MS/MS) system (ACQUITY UPLC-Xevo TQ-S, Waters Corp., Milford, MA) was used to quantitate bile acids in the human plasma/urine samples based on previously published protocols (Xie, Zhong et al. 2013, Ferslew, Xie et al. 2015). Data acquisition was performed using MassLynx version 4.1 and bile acid quantification was performed using the TargetLynx applications manager version 4.1 (Waters, Milford, MA).

### Statistical Analysis

Chemometric analysis was performed on metabonomics data using the software package SIMCA-P+ (version 16.0, Sartorius Stedim Biotech, Sweden). Principal component analysis (PCA) and a modification of Partial Least Squares Regression (PLSR) that removes all information orthogonal to the response variable during the fitting process were employed. This variant, Orthogonal Projection to Latent Structures (O-PLS) (Trygg and Wold 2003) provides sparser models (improving their interpretability) with the same degree of fit as PLSR. To highlight the weight of individual variables in the model, Variable Importance in Projection (VIP) was used, with a value above 1 used as a threshold by convention. Univariate analysis has been conducted using unpaired and paired t-test for group comparison, spearman correlations between metabolites and HAD, STAI, Amygdala endpoints and bacterial counts were computed. Statistical analysis was carried out using R 4.0.5 (2021 Mar. 31).

### Results

### Study Patients and Biological Samples

From the 38 study patients who completed the study (BL=18, placebo=20), metabonomics analysis of blood samples could be conducted on 36 participants for which samples were available at both pre- and post-intervention (BL=18, placebo=18). The quantity of BL in feces could be performed on 35 participants (BL=16, placebo=19),

BL Quantification in Fecal Samples & Association with Clinical Outcome

A high amount of B. longum was only detected in the probiotic group with exception of one subject in the placebo group, indicating good compliance with the intervention.

The reduction of two points or more in either HAD-A or HAD-D sub-scores, as success criteria of the trial, was associated with increased abundance of BL (p=0.0034 and p=0.0026, respectively). The reduction of both scores correlated with the abundance of BL in feces (rho=−0.4, p=0.018 and rho=0.55, p=6e−04, respectively). Decreased amygdala activation in response to negative emotional stimuli, measured by fRMI, also correlated with the BL abundance (rho=0.48, p=0.016).

### Treatment and Plasma Metabolic Phenotype

OPLS discriminant analysis was applied using one predictive and one orthogonal components to model blood metabolic differences between the two groups. The model was statistically robust only for post-treatment analysis (R²x=0.19, R²Y=0.76, Q²Y=0.26, where R²X: explained variance in the metabonomics data (urine metabolites), R²Y: explained group variance (placebo and probiotic) and Q²Y: robustness of the model). There was no differences between the two groups before treatment (Q²Y<0). From the most discriminant variables, statistically significant differences between groups were tested using paired t-test, and results are reported in Table 1 together with blood concentrations, and OPLS derived parameters. BL treated patients showed a higher blood concentration (p<0.1) in glycine-conjugated bile acid species (GCA, GCDCA), and lower blood concentration of the bile acid GUDCA (p=0. 0.108) (FIG. 1).

TABLE 1
Conjugated bile acids differences according to treatment and time.

| | OPLS | | Blood concentrations (Mean (SD)) | | | | |
|---|---|---|---|---|---|---|---|
| Metabolites | parameters | | | Placebo | | | P- |
| (unit) | Coeff | VIP | Placebo pre | post | BL Pre | BL Post | value |
| GCA (nM/L) | 0.34 | 1.23 | 331.2 (632.3) | 258.7 (158) | 385.2 | 602.2 (786.1) | 0.085 |
| | | | | | (291.2) | | |
| GCDCA (nM/L) | 0.32 | 1.23 | 1069.2 | 980.8 | 1371.9 | 1807.2 | 0.061 |
| | | | (1042.4) | (707.6) | (887.7) | (1632.6) | |
| GUDCA (nM/L) | −0.31 | 1.11 | 35.6 (61.1) | 69.7 | 54.9 (106) | 17.6 (23.4) | 0.108 |

Legend: Coeff: OPLS Correlation coefficient, VIP: OPLS Variable Importance in Projection; p-value: unpaired t-test between placebo and BL group post intervention.

| | Changes in amygdala activation | | | | Changes in HAD score | | | |
|---|---|---|---|---|---|---|---|---|
| | BL (N = 11) | | All patients (N = 23) | | BL (N = 18) | | All patients (N = 36) | |
| Metabolites | Rho | p-value | Rho | p-value | Rho | p-value | Rho | p-value |
| GCA | −0.13 | 0.714 | 0.13 | 0.552 | −0.06 | 0.808 | −0.01 | 0.964 |
| GCDCA | 0.01 | 0.989 | 0.18 | 0.182 | 0.04 | 0.866 | −0.04 | 0.828 |
| GUDCA | 0.25 | 0.451 | 0.52 | 0.012 | −0.11 | 0.668 | 0.15 | 0.385 |

TABLE 2
Correlation of plasma metabolites concentrations post-intervention with changes in
clinical endpoints and with fecal probiotic counts

| | Changes in HADA score | | | | BL count post intervention | | | |
|---|---|---|---|---|---|---|---|---|
| | BL (N = 18) | | All patients (N = 36) | | BL (N = 16) | | All patients (N = 33) | |
| Metabolites | Rho | p-value | Rho | p-value | Rho | p-value | Rho | p-value |
| GCA | 0.47 | 0.052 | 0.28 | 0.100 | 0.11 | 0.697 | 0.16 | 0.375 |
| GCDCA | 0.44 | 0.065 | 0.14 | 0.426 | 0.20 | 0.463 | 0.18 | 0.319 |
| GUDCA | 0.12 | 0.628 | 0.30 | 0.072 | 0.12 | 0.656 | −0.15 | 0.395 |

Notably, the post-intervention plasma concentration of two primary bile acids glycine-conjugated cholic acid (GCA, rho=0.47, p=0.052) and glycine-conjugated chenodeoxycholic acid (GCDCA, rho=0.44, p=0.065), was positively correlated with the improvement of anxiety symptoms in the IBS subject in the probiotic group (Table 2). Therefore, BL NCC3001 intake is postulated to increase the reabsorption of these bile acids in the gut, and to reduce glycine-deconjugation by other gut bacteria.

Gycine-conjugated bile acids, cholic acid, chenodeoxycholic acid are amongst the most abundant species involved in emulsification of dietary fats, and are increased with BL NCC3001. Prior to their secretion into bile, bile acids are conjugated with the amino acid taurine or glycine (Hofmann et al. 2010). Conjugation substantially reduces the passive reabsorption of the molecule through biological membranes. This allows bile acids in the intestine to form micelles, which are necessary for the absorption of lipids. Subsequently, a wide variety of intestinal microbes possess the ability to deconjugate bile acids, and these deconjugated forms are then returned to the liver for reconjugation (Hofmann et al. 2010). Several mode of actions could be mediated by the modulation of the bile acids composition exerted by probiotics such as BL NC3001, such as:
(i) influencing the bioavailability of glycine for normal neurotransmitter metabolism, and for which supplementation is associated with improved mood and cognition, and sleep (Bannai et al. Front Neurol. 2012; 3:61),
(ii) modulation of emulsification, absorption and bioavailability of dietary fats, including PUFAs, to cross blood brain barrier and contribute to brain function involved in mood, depression and anxiety (Kuan-Pin Su et al. Clin Psychopharmacol Neurosci. 2015 August; 13 (2): 129-137),
(iii) Modulation of the synthesis of Oleyl and palmitoyl ethanolamines lipids in the enterocytes, know to modulate Anxiety- and depressive-like behaviour as well as pain perception and sensory transduction (Hill M N et al., Psychoneuroendocrinology. 2009 September; 34 (8): 1257-62. And Coppola M, et al. Med Hypotheses. 2014 May; 82 (5): 507-11)., (iv) and by direct interaction with specific receptor such as FXR or, TGR5 receptors (Kiriyama Y, et al. Biomolecules. 2019).

1-2. (canceled)

3. Method for detecting and/or quantifying mood disorder, improvements of the mood disorder status and/or excessive emotional reaction of a subject, comprising
assessing the level of at least one conjugated bile acid in a body sample obtained from a subject to be tested, and
comparing the subject's conjugated bile acid level to a predetermined reference value, wherein:
i) an increased level of conjugated bile acid cholic acid (CA) and/or chenodeoxycholic acid (CDCA), in the sample compared to the predetermined reference value and/or
ii) a decreased level of conjugated bile acid ursodeoxycholic acid (UDCA), in the sample compared to the predetermined reference value,
indicates an improvement of the mood disorder status and/or excessive emotional reaction of the subject.

4. The method according to claim 3, wherein the mood disorder is mild to severe.

5. The method according to claim 3, wherein the predetermined reference value was obtained previously from the same subject.

6. The method according to claim 3, wherein the predetermined reference value is based on an average conjugated bile acids level in the same body fluid in a control population.

7. The method according to claim 3, wherein the levels of the biomarkers in the sample and in the reference are determined by mass spectrometry, according to by ultra-performance liquid or gas chromatography coupled to tandem mass spectrometry.

8. The method according to claim 3, wherein the predetermined reference value is based on at least one conjugated bile acid level obtained from the same body fluid as the level of conjugated bile acid in a body sample obtained from a subject to be tested.

9. The method according to claim 3, wherein the subject's predetermined reference value was obtained from a body sample that was collected from the subject before an intervention to treat or ameliorate a mood disorder status and/or excessive emotional reaction started.

10. The method according to claim 3, wherein the subject's predetermined reference value was obtained from a body sample that was collected from the subject during an intervention to treat or ameliorate a mood disorder status and/excessive emotional reaction, but at least one week, for example at least four weeks, before the body sample is obtained from the subject.

11. The method according to claim 3, wherein the body sample is selected from the group consisting of feces, whole blood, blood serum, and blood plasma.

12. The method according to claim 3, wherein the method is for monitoring the progress of an intervention to treat or ameliorate a mood disorder status and/or excessive emotional reaction in a subject, wherein the intervention comprises the administration of a probiotic.

13. The method according to claim 12, wherein the probiotic is BL NCC3001.

14. The method according to claim 3, wherein the subject is a human or a companion animal such as a cat or a dog.

15. An improved method to treat or ameliorate a mood disorder status and/or excessive emotional reaction in a subject in need, comprising administering to the subject, an effective amount of a composition combining a probiotic with glycine or a derivative thereof.

16. A method according to claim 15, wherein the mood disorder is mild to severe.

17. (canceled)

18. A method according to claim 15, wherein the glycine is in the form of L-glycine or any source rich in glycine.

19. A method according to claim 15, wherein the composition is administered orally.

20. A method according to claim 15, wherein the composition is a food product or beverage product.

21-22. (canceled)
